(19) United States
(12) Patent Application Publication
(10) Pub. No.: US 2014/0172074 A1
(43) Pub. Date: Jun. 19, 2014

(54) STENT WITH REINFORCED JOINTS

(71) Applicant: 480 BIOMEDICAL, INC., Watertown, MA (US)

(72) Inventors: Danny Concagh, Medfield, MA (US); Maria Palasis, Wellesley, MA (US); Yina Kuang, Newton, MA (US); Stephanie Webber, Brookline, MA (US); Kicherl Ho, Groton, MA (US); Wendy Naimark, Boston, MA (US)

(73) Assignee: 480 BIOMEDICAL, INC., Watertown, MA (US)

(21) Appl. No.: 13/713,302

(22) Filed: Dec. 13, 2012

(51) Int. Cl. A61F 2/90

(52) U.S. Cl. CPC A61F 2/90
USPC 623/1.19; 623/1.2

(57) ABSTRACT

The present invention relates generally to stents for insertion into bodily lumens that include at least one strand that forms a self-expanding tubular structure. Specifically, these strands form a plurality of intersections at which one portion of the strand is in proximity to another portion of the same or different strand. More specifically, at least some of those intersections include a plurality of reinforcement members.

### FIELD OF THE INVENTION

The present invention relates to implantable stents, and more particularly, to stents that include reinforcement members to enhance mechanical properties.

### BACKGROUND

A variety of medical conditions are treatable by the implantation of tubular devices into natural body lumens. For example, it is commonplace to implant balloon-expandable metallic stents into the coronary arteries of patients with heart disease following balloon angioplasty to minimize the risk that the arteries will undergo restenosis. Recently, commercial balloon-expandable stents have included drug-eluting polymer coatings that are designed to further decrease the risk of restenosis. Other examples of conventional tubular medical implants include woven grafts and stent-grafts that are used to span vascular aneurysms, polymeric tubes and catheters that are used to bypass strictures in the ureter and urethra, and stents that are used in the peripheral vasculature, prostate, and esophagus.

Despite the evolution of metallic stents, they continue to have limitations such as the possibility of causing thrombosis and vascular remodeling. While the use of biodegradable and biostable polymeric materials for stents and other implantable devices has been proposed to eliminate the possible long-term effects of permanent implants, the use of such materials has been hindered by relatively poor expandability and mechanical properties. For example, the expansion characteristics and radial strength of prototype stents made from biodegradable and biostable polymeric materials is often significantly lower than that of metallic stents. This is particularly the case where such stents are low profile and make use of small diameter fibers or thin walled struts that comprise the stent body. Furthermore, the degradation rate and the manner in which such devices degrade in the body may be difficult to control. Finally, where such devices are used as a drug delivery vehicle, the drug elution rate has been difficult to reproducibly characterize.

There is therefore a need for low-profile, self-expanding implantable tubular devices that have sufficient expansion characteristics, strength and other mechanical properties that are necessary to effectively treat the medical conditions for which they are used.

### SUMMARY OF THE INVENTION

In one aspect, the present invention comprises a stent for insertion into a bodily lumen. The stent comprises at least one strand that forms a self-expanding tubular structure. The strand or strands form a plurality of intersections at which a first portion of a strand is in proximity to another portion of the same or different strand. The stent further includes a plurality of reinforcement members, each of which are located at one of the intersections.

In another aspect, the present invention comprises a method of treating a patient by providing a stent in accordance with the present invention, and delivering the stent to a target location within the patient's body.

In another aspect, the present invention comprises a kit that includes the stent in accordance with the present invention and a delivery device useful for delivering the stent to a target location within a patient's body.

In certain embodiments, the reinforcement members comprise a quantity of polymer material. In certain other embodiments, the reinforcement members comprise rivets. In yet certain other embodiments, the reinforcement members comprise fibers wrapped about the stent strands.

### BRIEF DESCRIPTION OF THE DRAWINGS

FIG. 1 is a side view of a woven stent suitable for use with the present invention.

FIG. 2 is a side view of a non-woven stent suitable for use with the present invention.

FIG. 3a is a close-up view of the side of a woven stent with reinforcement members, in accordance with an embodiment of the present invention.

FIG. 3b is a cross-sectional view of an intersection of a woven stent with a reinforcement member, in accordance with an embodiment of the present invention.

FIG. 4a is a close-up view of the side of a non-woven stent with a reinforcement member, in accordance with an embodiment of the present invention.

FIG. 4b is a cross-sectional view of an intersection of a non-woven stent with a reinforcement member, in accordance with an embodiment of the present invention.

FIG. 5a is a close-up view of the side of a woven stent with rivet reinforcement members, in accordance with an embodiment of the present invention.

FIG. 5b is a cross-sectional view of an intersection of a woven stent with a rivet reinforcement member, in accordance with an embodiment of the present invention.

FIG. 6a is a close-up view of the side of a woven stent with fiber winding reinforcement members, in accordance with an embodiment of the present invention.

FIG. 6b is a cross-sectional view of an intersection of a non-woven stent with a fiber winding reinforcement member and a polymer reinforcement member, in accordance with an embodiment of the present invention.

### DETAILED DESCRIPTION OF THE PREFERRED EMBODIMENTS

The present invention provides for self-expanding stents that have expansion characteristics and mechanical properties that render them suitable for a broad range of applications involving placement within bodily lumens or cavities. When compared with conventional self-expanding stents, the stents of the present invention recover to an exceptionally high percentage of their manufactured diameter after being crimped and held in a small diameter for delivery into a bodily lumen. Moreover, when compared with conventional self-expanding stents and particularly polymeric stents, the stents of the present invention are characterized by much improved strength and other desired mechanical properties. These objectives are achieved through the use of reinforcement members at the intersection of stent strands, which act to limit the movement of the strands relative to each other at the location of such intersections.

As used herein, “stent” is used synonymously with scaffolds, endoprostheses or other substantially tubular structures that may be implanted into the human body. Further, although the present invention is described with specific reference to stents, it may be applied to any suitable implantable materials and structures. The stents of the present invention are described to comprise “strands,” which, as used herein, include fibers, extruded elements, struts and other flexible and inflexible elements formed by any suitable method that are moveable with respect to each other in the absence of the reinforcements members of the present invention. The one or more strands of the stents of the present invention are said to be in “proximity” to each other, meaning that they are in physical contact or sufficiently close to being in physical contact so that they may be connected to each other with the disclosed reinforcement members. Also as used herein, “self-expanding” is intended to include devices that are crimped to a reduced configuration for delivery into a bodily lumen or cavity, and thereafter tend to expand to a larger suitable configuration once released from the delivery configuration, either without the aid of any additional expansion devices or with the partial aid of balloon-assisted or similarly-assisted expansion. As used herein, “strength” and “stiffness” are used synonymously to mean the resistance of the stents of the present invention to deformation by radial forces. The term “bioabsorbable” is used herein synonymously with “biodegradable” and “bioerodible” to describe a material or structure that degrades in the human body by any suitable mechanism. As used herein, “woven” is used synonymously with “braided.”

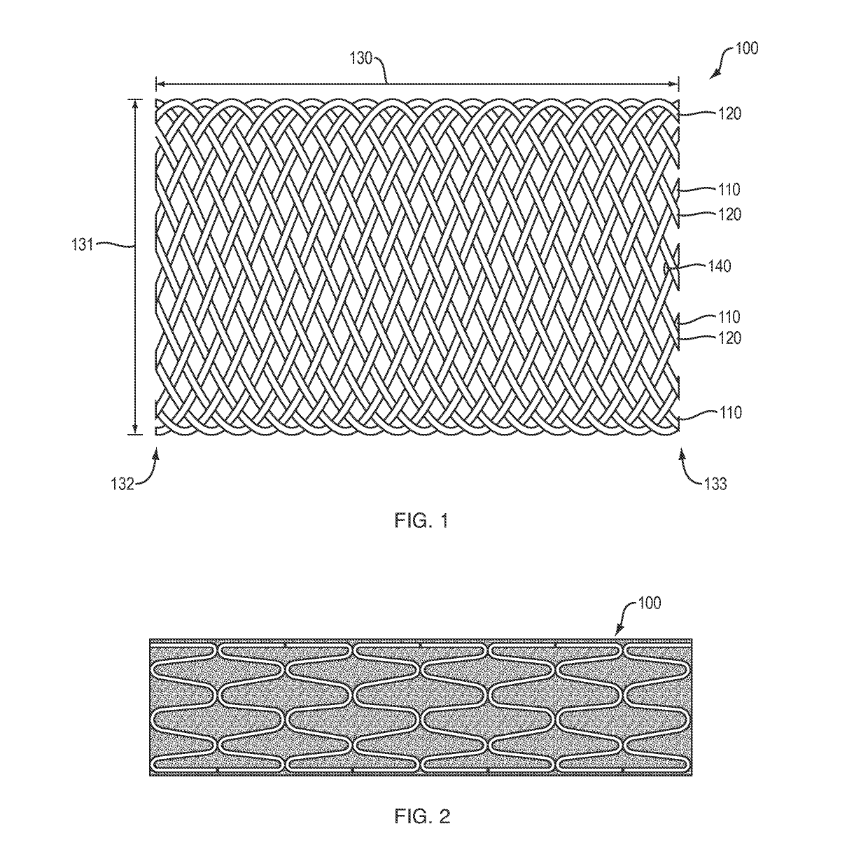

The stent structures and related technology suitable for use with the present invention are described in U.S. Ser. No. 13/370,025, which is incorporated herein by reference for all purposes. In one embodiment shown in FIG. 1, the stent 100 preferably comprises at least one strand woven together to form a substantially tubular configuration having a longitudinal dimension 130, a radial dimension 131, and first and second ends 132, 133 along the longitudinal dimension. For example, the tubular configuration may be woven to form a tubular structure comprising two sets of strands 110 and 120, with each set extending in an opposed helix configuration along the longitudinal dimension of the stent. The sets of strands 110 and 120 cross each other at a braid angle 140, which may be constant or may change along the longitudinal dimension of the stent. Preferably, there are between about 16 and about 96 strands used in the stents of the present invention, and the braid angle 140 is within the range of about 90 degrees to about 135 degrees throughout the stent. The strands are woven together using methods known in the art, using known weave patterns such as Regular pattern “1 wire, 2-over/2-under”, Diamond half load pattern “1 wire, 1-over/1-under”, or Diamond pattern “2 wire, 1-over/1-under”.

Although the strands may be made from biostable polymeric or metallic materials, they are preferably made from at least one biodegradable polymer that is preferably fully absorbed within about two years of placement within a patient, and more preferably within about one year of placement within a patient. In some embodiments, the strands are fully absorbed within about twelve or fewer months of placement within a patient. The first and second strand sets 110, 120 may be made from the same or different biodegradable polymer. Non-limiting examples of biodegradable polymers that are useful in the at least one strand of the present invention include poly lactic acid (PLA), poly glycolic acid (PGA), poly trimethylene carbonate (PTMC), poly caprolactone (PCL), poly dioxanone (PDO), and copolymers thereof. Preferred polymers are poly(lactic acid co-glycolic acid) (PLGA) having a weight percentage of up to about 20% lactic acid, or greater than about 75% lactic acid (preferably PLGA 85:15), with the former being stronger but degrading in the body faster. The composition of PLGA polymers within these ranges may be optimized to meet the mechanical property and degradation requirements of the specific application for which the stent is used. For desired expansion and mechanical property characteristics, the materials used for the strands preferably have an elastic modulus within the range of about 1 to about 10 GPa, and more preferably within the range of about 6-10 GPa.

To facilitate the low-profile aspects of the present invention (e.g., the delivery of the stents into small diameter bodily lumens or cavities), the strands used in the stent 100 preferably have a diameter in the range of from about 125 microns to about 225 microns, and are more preferably less than about 150 microns in diameter. The use of small diameter strands results in an stent with minimal wall thickness and the preferred ability to collapse (i.e., to be crimped) within low diameter catheter delivery systems. Where multiple strands are used, they may be of substantially equal diameters within this range, or first strand set 110 may be of a different general diameter than second strand set 120. In either event, the diameters of strands are chosen so as to render the stent 100 preferably deliverable from a 10 French delivery catheter (i.e., 3.3 mm diameter) or smaller, and more preferably from a 7 French delivery catheter (i.e., 2.3 mm diameter) or smaller. The ability to place the stent of the present invention into small diameter delivery catheters allows for its implantation into small diameter bodily lumens and cavities, such as those found in the vascular, biliary, uro-genital, iliac, and tracheal-bronchial anatomy. Exemplary vascular applications include coronary as well as peripheral vascular placement, such as in the superficial femoral artery (SFA). It should be appreciated, however, that the stents of the present invention are equally applicable to implantation into larger bodily lumens, such as those found in the gastrointestinal tract. It should also be appreciated that for use within larger bodily lumens such as the esophagus, duodenum, colon, etc., the stent may be scaled to a larger diameter that preferably makes use of fiber diameters larger than about 225 microns.

In another embodiment of the present invention, the stent is a non-woven, self-expanding structure, such as a unitary polymeric framework. As shown in FIG. 2, the non-woven stent 100 is preferably characterized by a regular, repeating pattern such as a lattice structure. The use of a unitary framework may provide a reduced profile when compared to the use of woven strands, which yield a minimum profile that is the sum of the widths of overlapping strands. In addition, a unitary framework eliminates the possible change in length of the stent associated with crimping and subsequent expansion, known as foreshortening, which is common in braided stents. When the stent 100 is a unitary framework, it is fabricated using any suitable technique, such as by laser cutting a pattern into a solid polymer tube. In a preferred embodiment, when the stent 100 is a unitary framework, it is formed by laser cutting and includes a wall thickness of between about 75 and about 100 microns. It should be recognized that while the present invention is described primarily with reference to woven strand configurations, aspects of the present invention are equally applicable to non-woven, self-expanding structures unless necessarily or expressly limited to woven configurations.

All embodiments of the present invention include reinforcement members. The reinforcement members are located at the intersections formed by the strand(s) used to form the stent 100. The intersections are defined by locations at which strands are in proximity to each other such that they are in physical contact or sufficiently close to being in physical contact so that they may be connected to each other with the reinforcement members.

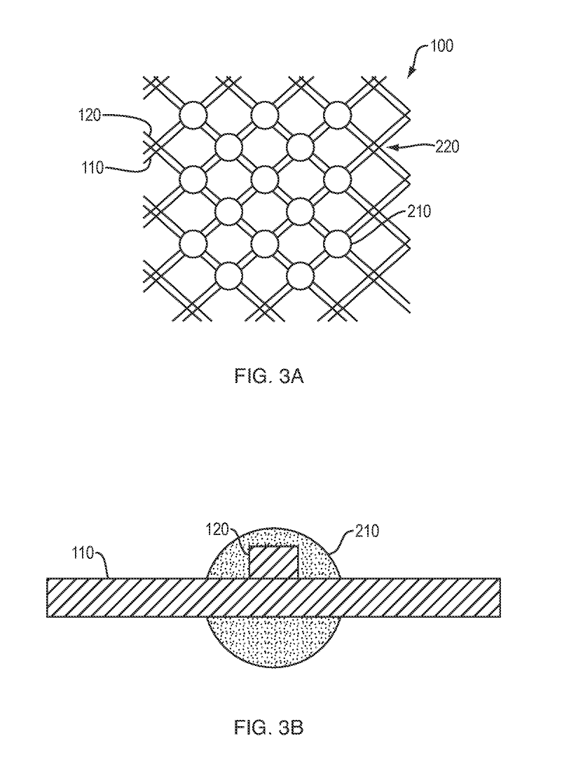

In one embodiment of the invention shown in FIGS. 3a and 3b, reinforcement members 210 are placed at some or all of the intersections 220 of the stent 100. It should be noted that only a portion of stent 100 is shown in FIGS. 3a and 3b, as well as in subsequent figures. In this embodiment, the stent 100 is woven as described with reference to FIG. 1, in which the stent 100 may be fabricated from a single or multiple strands 110, 120. As such, the intersections 220 are locations at which strands 110, 120 overlap each other and make physical contact, as shown in the cross-sectional view shown in FIG. 3b. The reinforcement members 210 preferably comprise a quantity of polymeric material that the portions of the strand(s) 110, 120 that are in proximity to each other. In other words, the reinforcement members 210 coat the strands(s) 110, 120 in the vicinity of the intersections 220 and do not coat the strand(s) 110, 120 in locations that are not in the vicinity of the intersections 220. In various embodiments, the polymer material that constitutes the reinforcement members extend no more than 1 mm, 0.5 mm, 250 microns, 100 microns, or 50 microns from the center of the intersections 220. Preferably, the reinforcement members 210 coat the strand(s) 110, 120 such that they are completely surrounded or encapsulated by the reinforcement members 210 in the vicinity of the intersections 220, as shown in FIG. 3b. In other embodiments, the reinforcement members 210 only partially surround the strand(s) 110, 120. In still other embodiments, the reinforcement members comprise polymeric material such as an elastomeric adhesive placed between strands where they intersect. In either event, however, the objective is that the strands will be substantially fixed at the intersections 220 that include reinforcement members 210 to hold the braid angle substantially constant through the crimping and expansion of the stent 100. The use of such reinforcements only at the intersections 220 of the stent 100 offers many advantages over fully coated stents, such as the use of less material that allows for smaller stent profiles, the ability to crimp the stent to smaller dimensions, the providing of stents with lower weight, and the avoidance of integrity problems that are sometimes encountered with coated stents such as the cracking and/or spallation of the coating material.

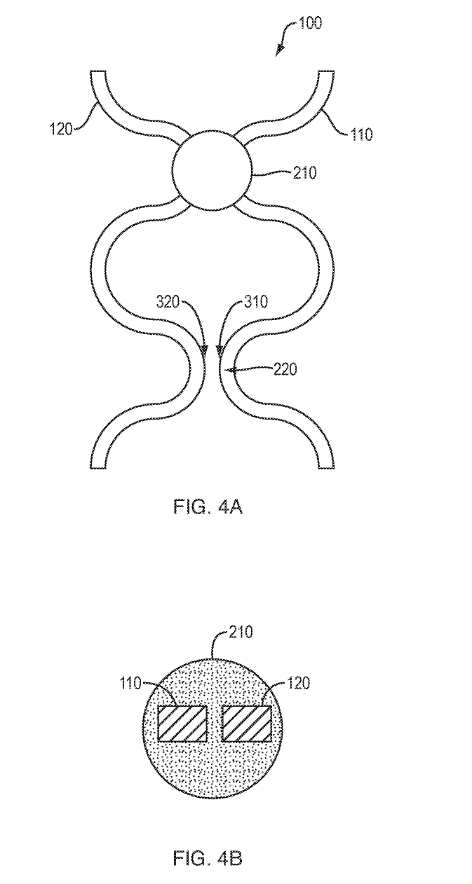

FIG. 4 illustrates a close-up of a non-braided stent 100 formed by laser cutting or the like. The strands 110, 120 are undulating elements that come into proximity to each other at peaks 310, 320 to form intersection 220. Although the strands 110, 120 do not come into physical contact with each other (as can be seen in the cross-sectional view shown in FIG. 4b), they are sufficiently close to being in physical contact so that they may be connected to each other with reinforcement members 210.

In the embodiments shown in FIGS. 3 and 4, the reinforcement members 210 comprise a suitable material, preferably a polymeric material and more preferably an elastomeric polymer material, applied to at least some of the intersections 220 of the stent 100. In some embodiments, the reinforcement members 210 are applied to all of the intersections 220 of the stent 100. The reinforcement members 210 are applied by any suitable technique, such as ink jet printing techniques as described in U.S. Pat. No. 7,709,048, which is incorporated herein by reference for all purposes. Suitable materials for use as reinforcement members 210 include biocompatible metallic, polymeric and ceramic materials that may be biostable or bioabsorbable. Non-limiting examples of such materials include TECOTHANE® (Lubrizol Advanced Materials, Inc.), elastane, PELLETHANE® (Lubrizol Advanced Materials, Inc.), poly (styrene-b-isobutylene-b-styrene (SIBS), poly(lactic acid-co-caprolactone) (PLCL), poly(glycolide-co-caprolactone) (PGCL), and poly(lactic acid-co-dioxanone) (PLDO), certain homopolymers such as poly trimethylene carbonate (PTMC), and copolymers and terpolymers thereof. Other suitable materials for use as reinforcement members include shape memory materials, such as shape memory alloys such as nitinol and shape memory polymers, that have a shape transition temperature below body temperature (i.e., 37° C.) such that the reinforcement members undergo a shape transformation upon placement in the body that urges the stent 100 towards its expanded configuration after being crimped to some smaller configuration for insertion into the body.

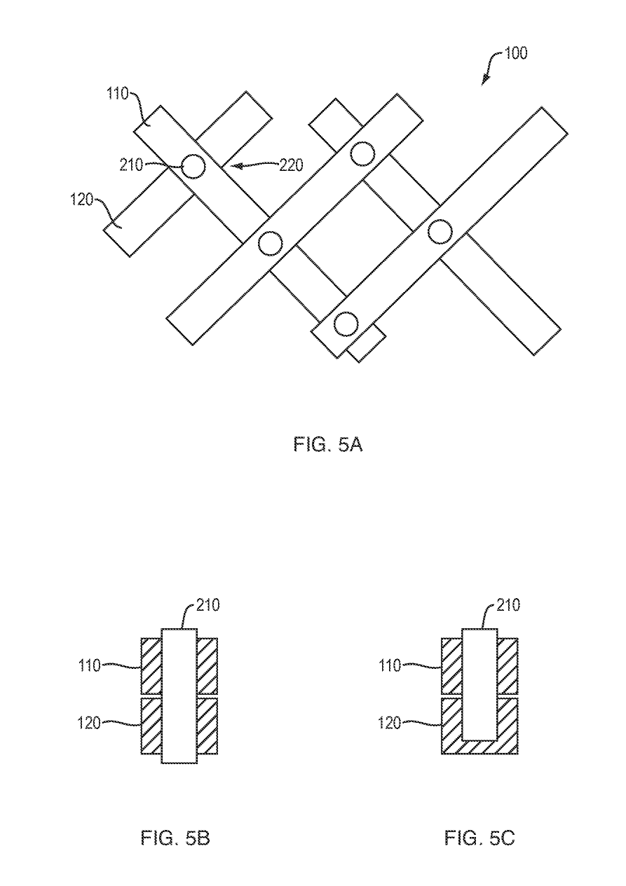

In another embodiment shown in FIGS. 5a and 5b, the reinforcement members 210 of the present invention comprise rivets that extend through the strand(s) 110, 120 at intersections 220 where portions of the strands are in proximity to each other. In the embodiment shown in FIGS. 5a and 5b, the stent 100 is woven such that the strand(s) 110, 120 are in physical contact. It should be appreciated, however, that this embodiment is equally applicable to embodiments in which the stent is non-woven. In this embodiment, the reinforcement members 210 have a length that is greater than the width of one of the strands such that they extend completely through one of the strands and at least partially through the other strand. In a preferred embodiment, the reinforcement members 210 have a length that is greater than the width of both of the strands at the intersections 220 such that the reinforcement members 210 extend completely through both strand portions, as shown in the cross-sectional view of FIG. 5b. The reinforcement members 210 are made from any suitable biocompatible metallic, polymeric and ceramic materials that may be biostable or bioabsorbable, such as the materials listed for use in the embodiments shown in FIGS. 3 and 4. In some embodiments, the rivets are elastomeric or are otherwise deformable to help facilitate the compressibility of the stent 100 into a delivery catheter. In addition, the reinforcement members 210 in this embodiment may optionally include roughened surfaces or protruding members that extend into the strand(s) 110, 120 to help to achieve the objective of keeping the positions of the strand(s) 110, 120 substantially fixed at the intersections 220 that include reinforcement members 210 to hold the braid angle substantially constant through the crimping and expansion of the stent 100. In other embodiments, the rivets are coated with a material that has a high coefficient of friction with respect to the materials used in strand(s) 110, 120.

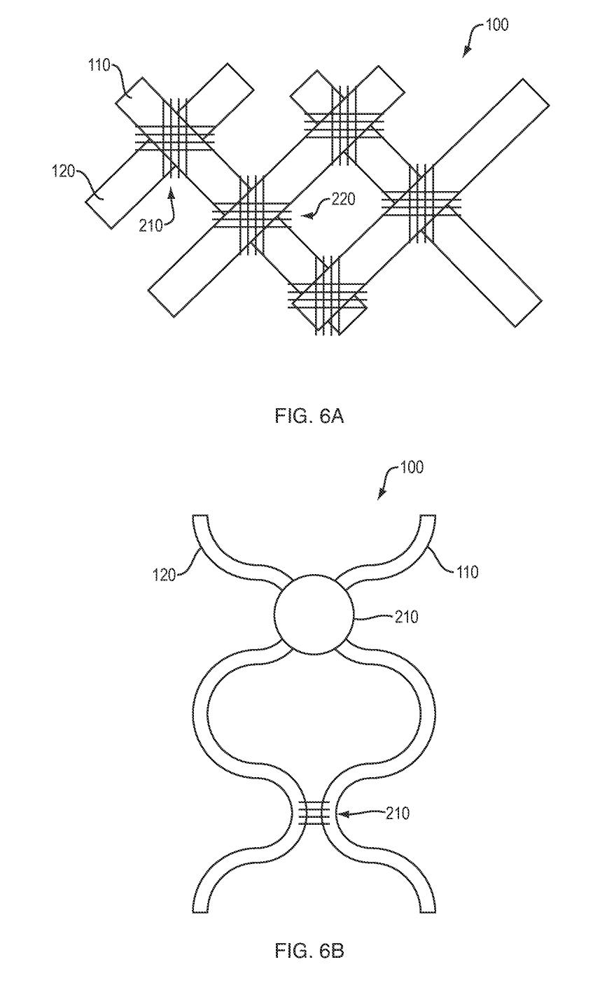

In another embodiment, the reinforcement members 210 of the present invention comprise fibers that are wrapped around at least some of the intersections of the stent 100, as shown in FIGS. 6a and 6b. In this embodiment, the fibers are any suitable biocompatible metallic, polymeric or ceramic material that is extruded or otherwise formed into an elongated structure that can be wrapped around the intersections 220. The fibers 270 are preferably an elastomeric polymer material, such as the materials listed above for the embodiments shown in FIGS. 3 and 4. As used herein, “fiber” refers to any elongated structure or bands that may be used for the purposes described herein. As shown in FIG. 6a, the fibers may be wrapped in directions that are both parallel and orthogonal to the longitudinal axis of the stent, or they may be wrapped in either (but not both) of these directions. The fibers 270 provide forces upon the strand(s) 110, 120 that act to hold the strands substantially fixed at the intersections 220 that include reinforcement members 210 to hold the braid angle substantially constant through the crimping and expansion of the stent 100. Alternatively, the fibers are held in compression when the stent is in a crimped configuration (such as when the fibers are oriented in a direction parallel to the longitudinal direction of the stent), and expand when the stent is released from the crimped configuration to help self-expansion of the stent. FIG. 6a illustrates the applicability of fibers 270 as reinforcement members 210 in a woven stent, and FIG. 6b illustrates the applicability of fibers 270 as reinforcement members 210 in a non-woven stent. FIG. 6b also illustrates an embodiment of the present invention in which different types of reinforcement members are used in a single stent.

The stent 100 of the present invention is self-expanding in that it is manufactured at a first diameter, is subsequently reduced or “crimped” to a second, reduced diameter for placement within a delivery catheter, and self-expands towards the first diameter when extruded from the delivery catheter at an implantation site. The first diameter is preferably at least 20% larger than the diameter of the bodily lumen into which it is implanted. The stent 100 is preferably designed to recover at least about 80%, more preferably at least about 90%, and up to about 100% of its manufactured, first diameter.

The stents of the present invention are preferably radiopaque such that they are visible using conventional fluoroscopic techniques. In one embodiment, radiopaque additives are included within the polymer material of one or more strands of stent 100. Examples of suitable radiopaque additives include particles comprising iodine, bromine, barium sulfate, and chelates of gadolinium or other paramagnetic metals such as iron, manganese, or tungsten. In another embodiment, the radiopaque groups, such as iodine, are introduced onto the polymer backbone. In yet another embodiment, one or more biostable or biodegradable radiopaque markers, preferably comprising platinum, iridium, tantalum, and/or palladium are produced in the form of a tube, coil, sphere, or disk, which is then slid over one or more strands of fiber to attach to the ends of stent 100 or at other predetermined locations thereon. When the marker is in the form of a tube or coil, it has a preferable wall thickness of about 0.050 to 0.075 mm and a length of about 0.3 to 1.3 mm. The tube is formed by extrusion or other methods known in the art. The coil is formed by winding a wire around a mandrel of desired diameter and setting the coil with heat or other methods known in the art.

Embodiments of the present invention include stents that are covered with nonporous, nanoporous or microporous coverings.

To facilitate delivery, the stent 100 is preferably loaded into a delivery catheter just prior to being implanted into a patient. Loading the stent 100 in close temporal proximity to implantation avoids the possibility that the polymer of the stent 100 will relax during shipping, storage, and the like within the delivery catheter and therefore cannot fully expand to a working configuration. As such, one aspect of the invention includes a method of delivering a stent of the invention that comprises the step of loading the stent into a delivery catheter within a short period of time, and preferably within one hour, before implantation into a body lumen. It should be noted, however, that it is not required that the stents of the present invention are loaded into delivery catheters just prior to being implanted. In fact, one advantage of the present invention is that it provides self-expanding implantable medical devices with preferred expansion characteristics and mechanical properties even after being loaded in a delivery catheter for prolonged periods.

The present invention provides woven and non-woven self-expanding stents for placement within a bodily lumen that have sufficient strength and other mechanical properties that are necessary to effectively treat a variety of medical conditions. While aspects of the invention have been described with reference to example embodiments thereof, it will be understood by those skilled in the art that various changes in form and details may be made therein without departing from the scope of the invention.

1. A stent for insertion into a bodily lumen, comprising:
at least one strand forming a self-expanding tubular structure, the at least one strand forming a plurality of intersections in which, at each intersection, a first portion of said at least one strand is in proximity to a second portion of said at least one strand; and
a plurality of reinforcement members, each of said plurality of reinforcement members being located at a respective one of said intersections.

2. The stent of claim 1, wherein said reinforcement members comprise a quantity of polymer material that coat said first and second portions of said at least one strand.

3. The stent of claim 2, wherein said polymer material is an elastomer.

4. The stent of claim 2, wherein said polymer material coats said at least one strand only at said first and second portions of said at least one strand.

5. The stent of claim 4, wherein said polymer material of said reinforcement members and the at least one strand both comprise at least one biodegradable material.

6. The stent of claim 1, wherein said reinforcement members comprise rivets that extend through said first and second portions of said at least one strand.

7. The stent of claim 6, wherein said rivets each comprise a rod having a length that is greater than a width of said first portion plus a width of said second portion of said at least one strand.

8. The stent of claim 6, wherein said rivets each comprise a rod having a length that is less than a width of said first portion plus a width of said second portion of said at least one strand.

9. The stent of claim 6, wherein said rivets and the at least one strand both comprise at least one biodegradable material.

10. The stent of claim 6, wherein said rivets comprise a roughened surface.

11. The stent of claim 1, wherein said reinforcement members comprise fibers that are wrapped around the at least one strand at said plurality of intersections.

12. The stent of claim 11, wherein said fibers comprise an elastomeric material.

13. The stent of claim 1, wherein said reinforcement members comprise a shape memory material having a shape transition temperature less than 37° C.

14. The stent of claim 1, wherein said reinforcement members comprise a material having a glass transition temperature less than 37° C.

15. The stent of claim 1, wherein said at least one strand and said plurality of reinforcement members both comprise biostable materials.

16. The stent of claim 1, wherein said at least one strand and said plurality of reinforcement members both comprise biodegradable materials.

17. A stent for insertion into a bodily lumen, comprising:
at least one strand forming a self-expanding tubular structure, the at least one strand forming a plurality of intersections in which, at each intersection, a first portion of said at least one strand is in proximity to a second portion of said at least one strand; and
a plurality of reinforcement members, each of said plurality of reinforcement members being located at a respective one of said intersections;
wherein said reinforcement members comprise a quantity of polymer material that coat said first and second portions of said at least one strand only at said first and second portions of said at least one strand.

18. A stent for insertion into a bodily lumen, comprising:
at least one strand forming a self-expanding tubular structure, the at least one strand forming a plurality of intersections in which, at each intersection, a first portion of said at least one strand is in proximity to a second portion of said at least one strand; and
a plurality of reinforcement members, each of said plurality of reinforcement members being located at a respective one of said intersections;
wherein said reinforcement members comprise rivets that extend through said first and second portions of said at least one strand, wherein said rivets each comprise a rod having a length that is greater than a width of said first portion plus a width of said second portion of said at least one strand.
